(19) United States
(12) Patent Application Publication
(10) Pub. No.: US 2025/0346629 A1
(43) Pub. Date: Nov. 13, 2025

(54) USE OF D-ENANTIOMERIC PEPTIDE LIGANDS OF MONOMERIC ALPHA-SYNUCLEIN FOR THE THERAPY OF VARIOUS SYNUCLEINOPATHIES

(71) Applicant: PRIAVOID GMBH, Düsseldorf (DE)

(72) Inventors: Dieter WILLBOLD, Juelich (DE); Jeannine MOHRLUEDER, Erkelenz (DE); Marc SEVENICH, Koeln (DE); Tim ALTENDORF, Wuerselen (DE); Karoline Bianka SANTUR-DABOW, Koeln (DE)

(21) Appl. No.: 18/282,591

(22) PCT Filed: Mar. 22, 2022
(86) PCT No.: PCT/EP2022/057456

§ 371 Date: Sep. 18, 2023

(30) Foreign Application Priority Data
Mar. 22, 2021 (DE) 10 2021 107 061.9

(51) Int. Cl. C07K 7/08; A61K 38/00

(52) U.S. Cl. CPC C07K 7/08; A61K 38/00

(57) ABSTRACT

The invention relates to a peptide, comprising an amino acid sequence selected from the group consisting of SEQ ID NO: 1, SEQ ID NO: 2, SEQ ID NO: 3, SEQ ID NO: 4, SEQ ID NO: 5, SEQ ID NO: 6 or SEQ ID NO: 7 and also to such a peptide for use in the treatment of synucleinopathies.

### SEQUENCE LISTING

The instant application contains a Sequence Listing which has been submitted electronically in ASCII format and is hereby incorporated by reference in its entirety. Said ASCII copy, created on Apr. 1, 2024, is named P50812_SL_corr and is 1,775 bytes in size.

### BACKGROUND OF THE INVENTION

### 1. Field of the Invention

The invention relates to a peptide, comprising an amino acid sequence selected from the group consisting of SEQ ID NO: 1, SEQ ID NO: 2, SEQ ID NO: 3, SEQ ID NO: 4, SEQ ID NO: 5, SEQ ID NO: 6 or SEQ ID NO: 7, homologs, fragments and portions thereof, and also to such a peptide for use in the treatment of synucleinopathies.

### 2. Discussion of Background Information

Synucleinopathies cover a heterogeneous group of neurodegenerative diseases such as Parkinson's disease (PD), dementia with Lewy bodies (DLB) and multiple system atrophy (MSA), which are associated with the misfolding and aggregation of the protein α-synuclein in certain cells.

Insoluble inclusion bodies found in affected neurons of PD patients contain amyloid forms of α-synuclein, suggesting a causal relationship between amyloid formation and the disease pathology. It is assumed that α-synuclein aggregates in various synucleinopathies correspond to different conformational states of α-synuclein which multiply in the manner of prions and are transferred from cell to cell.

According to data from the National Institute of Neurological Disorders and Stroke (https://www.ninds.nih.gov/), there are currently no substances able to cure or effectively halt PD, DLB and MSA. Nevertheless, there are a number of active ingredients which can be used to treat the respective symptoms.

In summary, a causal and significantly life-lengthening therapy is currently not available for most, if not all, synucleinopathies, and is urgently needed.

The object of the present invention was therefore that of developing new chemical entities which can disassemble toxic α-synuclein aggregates that are already present into native functional α-synuclein monomers, thereby making the therapeutic use of these chemical entities possible in various synucleinopathies.

The chemical entity or variants thereof to be used in the therapy is intended to bind to the native, endogenous α-synuclein protein with as great an affinity and specificity as possible, and thereby stabilize it. As a result, the balance between misfolded and natively folded α-synuclein conformations is shifted in favor of the latter. Thus, in an ideal case, α-synuclein oligomers and fibrils that are already present can be broken down into their monomeric constituents and thereby eliminated.

### SUMMARY OF THE INVENTION

This object is achieved by a peptide according to claim 1, in particular by a peptide comprising an amino acid sequence selected from the group consisting of SEQ ID NO: 1, SEQ ID NO: 2, SEQ ID NO: 3, SEQ ID NO: 4, SEQ ID NO: 5, SEQ ID NO: 6 or SEQ ID NO: 7, and also homologs, fragments and portions thereof.

Further preferred embodiments are defined in the dependent claims.

Hereinafter, “comprise” is intended to also cover “consist of”.

The peptides of SEQ ID NO: 1, SEQ ID NO: 2, SEQ ID NO: 3, SEQ ID NO: 4, SEQ ID NO: 5, SEQ ID NO: 6 or SEQ ID NO: 7 were found using an optimized mirror-image phage display selection.

It should be noted that the method of mirror-image phage display, alongside the selection of specific monomeric binders as described here, can also be used to find specific oligomeric binders or even to find ligands for other species that form in protein-misfolding diseases.

In mirror-image phage display, for example, a recombinant library of randomized peptide sequences, presented on the gp3 protein of the M13 phage and encoded in the genome thereof, is selected against the exact mirror image (D-enantiomer) of a naturally occurring L-enantiomeric target molecule (e.g. α-synuclein). The gp3 molecule, also known as gene product 3, is a protein in the phage capsid which is required for contact with the host cell.

The peptide sequence is advantageously presented at the N-terminus of the gp3 protein of the M13 phage and is encoded in the genome thereof.

The DNA sequence of the p3 gene of a selected phage is linked to the DNA sequence which contains the genetic information about the corresponding peptide sequence on the gp3 molecule, as a result of which this can be sequenced. After sequencing, the genome sequence can be translated into an amino acid sequence and synthesized as a D-enantiomeric peptide which binds to the physiological, L-enantiomeric, form of the target molecule (e.g. α-synuclein). All of the phages presenting different peptides on their surface as fusion proteins with gp3 are hereinafter referred to as phage library. The corresponding peptides represent the biomolecules to be selected in the experiment.

What are referred to as “biopanning” rounds (selection rounds) can be carried out, for example three rounds. In the process, the phage library is brought into contact with a fixed target molecule, also referred to as bait, and binding phages are isolated from the billions of other non-binding phages also present.

By way of example, the number of phages which preferentially bind to oligomeric or fibrillary species of α-synuclein is reduced by not offering these very species as bait. Phages with an increased affinity for α-synuclein oligomers and fibrils can thereby be removed from the phage pool, meaning that, for example, α-synuclein monomer-specific phages accumulate. The method can of course be adapted analogously in order to specifically determine α-synuclein oligomer-binding ligands and peptides.

In order to reduce any accumulation of phages with an affinity for plastic, BSA or streptavidin, according to the invention, use is additionally made of different substrate surfaces, preferably in all biopanning rounds. In this case, a substrate surface is defined as a combination of the biotinylated substrate used (polystyrene or polypropylene surface derivativized with streptavidin) and the blocking or quenching agents used. In the successive selection rounds, the selection pressure is gradually increased. To this end, while the concentration of the target molecule (e.g. monomeric α-synuclein) remains stable, the number of washing steps after phage incubation is increased starting from the 2nd selection round, so as not to remove phages with an affinity for α-synuclein monomers.

Furthermore, a different substrate surface is chosen in every selection round of the phage display, by using other agents for blocking the surface (e.g. BSA, milk powder, no blocking) after the target molecule has been immobilized on the substrate.

By way of example, there may be a changeover between a BSA-blocked polystyrene surface in round 1, a milk powder-blocked polypropylene surface in round 2, and a BSA-blocked polystyrene surface in round 3.

The changeover between different substrate surfaces increases the specificity for the target molecule or the bait relative to the surface. Moreover, this leads to a reduction in ligands that bind non-specifically to plastic surfaces, BSA or other components of the substrate surface other than the bait molecule.

In parallel to the actual phage display selection, control selections may for example be carried out, which are identical to the main selection but with the significant difference that no bait is used here. Data analysis of the sequences resulting from the control selections makes it possible to identify peptides which accumulate during the selection even in the absence of bait, and which are therefore irrelevant for all subsequent steps.

The method is therefore characterized by the following steps:
a) providing an immobilized bate on a substrate surface
b) bringing the immobilized molecule acting as bait into contact with a solution which contains a library of molecules to be selected
c) bringing the immobilized bait surrounded by the molecules into contact with a washing solution
d) separating and multiplying the molecules still bound to the bait after the immobilized bait surrounded by the molecules has been brought into contact with washing solution
e) repeating the stated steps, with a different substrate being used for each repetition
f) identifying the sequence of the molecules remaining on the bait after the repetition.

A different substrate is used e.g. by changing the type of substrate and/or by blocking or not blocking the substrate by means of reagents.

As bait, use is made of a molecule from the group consisting of proteins, peptides, RNA, DNA, mRNA and chemical compounds. As bait, use is in particular made of monomeric α-synuclein.

As the surface on which the bait is immobilized, use is for example made of a component from the group consisting of microtitration plates, magnetic particles, agarose beads or sepharose beads.

The bait according to point a) is therefore a compound to which the biomolecule to be selected is bound. According to the methods known from the prior art, it is fixed to a first surface. By way of nonlimiting examples, mention may be made, as bait, of proteins, peptides, RNA or DNA molecules, in particular α-synuclein monomers. As possible surfaces, use may for example be made of microtitration plates, magnetic particles, agarose beads or sepharose beads. The surface with the immobilized bait can subsequently be quenched, the functional groups of the substrate being inactivated in the process. Moreover, the hydrophobic free surfaces remaining on the substrate can be blocked with suitable agents.

In the second step b), the immobilized bait is brought into contact with a randomized library of molecules—especially biomolecules. These biomolecules compete for binding to the bait. The randomized library is a mixture of a very large number, for example 10¹², but also 10⁴ or just 100, different molecules in a mixture. Such a library can for example consist in each case of peptides, proteins, DNA, RNA or mRNA which are bound to specific vehicles and which can bind to the bait. Vehicles include for example phages, polysomes or bacterial surfaces. The library can consist of artificial constituents or constituents isolated from nature, or a mixture of both. In the context of the invention, artificial means for example compounds produced from oligonucleotide synthesis.

In step c), the immobilized bait surrounded by biomolecules can be brought into contact with a washing substance. To this end, a washing step is carried out in step c), in which a buffer solution is brought into contact with the immobilized bait or is used to rinse same. This means that the solution with the library of biomolecules is preferably repeatedly replaced with an optionally similar or identical solution. This removes those library molecules that dissociate less quickly from the immobilized bait than other library molecules. The rate of the dissociation reaction of the binding library molecules is mainly determined by the different dissociation constants (in particular the k_{off} values) of the individual molecules. Thus, in statistical terms, those molecules with a small k_{off} value remain bound to the immobilized bait longest and therefore are statistically less likely to be washed away by the washing buffer. The liquid containing the washing buffer is preferably aqueous and may contain a pH buffer. Optional components of the solution for the specificity washing step may be salts, detergents or reducing agents.

After the specificity washing step in step c), in step d), the bound biomolecules are separated from the bait and multiplied. The separation or elution may for example be carried out by changing the pH, heating or other changes, in particular increasing the salt concentration. The separated or eluted biomolecules are subsequently multiplied using known methods. To this end, for example, phage particles which were obtained and eluted following steps a) to c) and which carry the biomolecules on their surfaces can be introduced into cells and multiplied.

In step e), the concentration of the selected biomolecules in the solution supplied to the bait after step a) is increased. Preferably, 3 to 6 selection rounds containing steps a) to e) are carried out. However, 1 to 10 or 1 to 20 repetitions can also be carried out. The increase in the competitor concentration, preferably carried out in step c), also leads to improved selection with an increasing number of cycles.

An increase in the washing steps, preferably carried out in step c), leads to improved selection with an increasing number of cycles.

A particularly relevant mirror-image phage display provides N-terminal biotinylated D-enantiomeric α-synuclein monomer in step a), a recombinant phage library in step b), and a buffer solution in step c), alongside α-synuclein monomer as bait. Elution as a separation step takes place, for example, by lowering the pH as a separation step and phage amplification as the multiplication in step d).

In this way, seven D-enantiomeric peptides that specifically bind α-synuclein monomer were developed.

SVD-1 (free N-terminus, amidated C-terminus):
kmpthetywqehiwha
SEQ ID NO 1:
SVD-6 (free N-terminus, amidated C-terminus):
SEQ ID NO 2:
ydwkqpmsasrflapw
SVD-10 (free N-terminus, amidated C-terminus):
SEQ ID NO 3:
sshwqqwnppywntds
SVD-14 (free N-terminus, amidated C-terminus):
SEQ ID NO 4:
ydwkqpmsasrflapwr
SVD-1a (free N-terminus, amidated C-terminus):
SEQ ID NO 5:
rlpthetywqehiwharrrrr
SVD-6a (free N-terminus, amidated C-terminus):
SEQ ID NO 6:
ydwrqplsasrflapwrrrrr
SVD-10a (free N-terminus, amidated C-terminus):
SEQ ID NO 7:
sshwqqwnppywntdsrrrrr

Sequence processing of SVD-1, SVD-6, SVD-10, SVD-14, SVD-1a, SVD-6a or SVD-10a (e.g. sequence variation) gives rise to the possibility of developing other substances that can be used therapeutically in synucleinopathies.

The present invention can also relate to other peptides that might have been identified using the above-disclosed method.

The peptides according to SEQ ID NO: 1 to 7 can be used as a potential medicine against synucleinopathies as a result of the specific binding to α-synuclein monomers.

The object according to the invention is also achieved by a peptide containing homologs, fragments and portions of the amino acid sequence according to SEQ ID NO: 1 to 7.

In this document, α-synuclein peptide or α-synuclein protein preferably means the human α-synuclein peptide or α-synuclein protein.

In the context of the invention, homologous sequences, or “homologs”, mean that an amino acid sequence has at least 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% identity to one of the abovementioned amino acid sequences of the monomers. Preference is given here to 80% and 90%. In the present description, the terms “homolog” and “homology” are used synonymously to “identity”. The identity between two nucleic acid sequences or polypeptide sequences is calculated by comparison, using the BESTFIT program based on the algorithm by Smith, T. F. and Waterman, M. S (Adv. Appl. Math. 2: 482-489 (1981)), adjusting the following parameters for amino acids: Gap creation penalty: 8 and Gap extension penalty: 2; and the following parameters for nucleic acids: Gap creation penalty: 50 and Gap extension penalty: 3. Preferably, the identity between two nucleic acid sequences or polypeptide sequences is defined by the identity of the nucleic acid sequence/polypeptide sequence over the same sequence length in each case, as is calculated by comparison using the GAP program based on the algorithm by Needleman, S. B. and Wunsch, C. D. (J. Mol. Biol. 48: 443-453), adjusting the following parameters for amino acids: Gap creation penalty: 8 and Gap extension penalty: 2; and the following parameters for nucleic acids: Gap creation penalty: 50 and Gap extension penalty: 3.

In the context of the present invention, two amino acid sequences are identical if they have the same amino acid sequence.

In a further variant, the peptides according to the invention have sequences which differ from the stated sequences by up to two or three amino acids.

Furthermore, sequences containing the above sequences can also be used as peptides.

The peptide according to the invention is further preferably characterized in that, at the free C-terminus, instead of the carboxyl group there is an acid amide group (CONH₂ group) or a COH group, COCl group, COBr group, CONH-alkyl radical or a CONH-alkylamine radical, or else the peptide is in cyclized form.

This particularly advantageously also achieves the object of providing a peptide without a negative charge at the C-terminus. This advantageously means that said peptide can bind to the target molecule with higher affinity than a peptide which has a carboxyl group at the free C-terminus. In the physiological state, peptides with a free unmodified carboxyl group have a negative charge at this end.

In one embodiment of the invention, the peptide according to the invention in the physiological state, in particular at a pH of 6-8, in particular at 6.5-7.5, in particular at pH 6.0, pH 6.1, pH 6.2, pH 6.3, pH 6.4, pH 6.5, pH 6.6, pH 6.7, pH 6.8, pH 6.9, pH 7.0, pH 7.1, pH 7.2, pH 7.3, pH 7.4, pH 7.5, pH 7.6, pH 7.7, pH 7.8, pH 7.9 or pH 8.0, is modified such that the C-terminus does not carry a negative charge and instead is neutral or has one or more positive charges.

In one embodiment, the peptide is characterized in that, at the free C-terminus, instead of the carboxyl group there is an acid amide group. Therefore, an acid amide group (—CONH₂ group) is arranged at the C-terminus instead of the carboxyl group (—COOH group).

Accordingly, the peptide is particularly advantageously amidated at the free C-terminus.

Accordingly, the peptide is particularly advantageously amidated at the free C-terminus and unmodified at the free N-terminus.

This particularly advantageously also achieves the object of having a peptide without excess negative charge which can bind with affinity to the target molecule, and which can be obtained in a simple manner.

In a further embodiment of the disclosure, the following further groups are present instead of the carboxyl group: COH, COCl, COBr, CONH-alkyl radical, CONH-alkylamine radical (net positive charge), etc., this being a nonlimiting list as long as the technical teaching of the main claim is followed.

In a further preferred embodiment of the invention, therefore, the binding affinity of the peptides modified according to the invention, without a negative charge at the C-terminus is increased, compared to linear peptides with a negative charge at the C-terminus but with an otherwise identical amino acid sequence, by 1%, 2, 3, 4, 5, 6, 7, 8, 9, in particular 10%, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, in particular 100%, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, in particular 200%, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, in particular 300%, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, in particular 400%, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, advantageously even 500%, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, particularly advantageously 600%, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, particularly advantageously 700%, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, likewise particularly advantageously 800%, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, likewise particularly advantageously 900%, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or even 1000%, or even 10 000%, or even up to 100 000% or 1 000 000%, it being possible to adopt any intermediate value.

This is indicated by a correspondingly lowered K_{D} value. The K_{D} value, as a measure of the binding affinity of a modified peptide to α-synuclein monomer, is lowered, compared to a linear binding peptide with a negative charge at the free C-terminus, by 1%, 2, 3, 4, 5, 6, 7, 8, 9, in particular 10%, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, in particular 99.1, 99.2, 99.3, 99.4, 99.5%, 99.6, 99.7, 99.8, 99.9 to 99.99 or even 99.999%, it being possible to adopt any intermediate value.

The peptide according to the invention is further preferably characterized in that it contains 2, 3, 4, 5, 6, 7, 8, 9, 10 or more copies of the sequences having SEQ ID NO: 1, SEQ ID NO: 2, SEQ ID NO: 3, SEQ ID NO: 4, SEQ ID NO: 5, SEQ ID NO: 6 and/or SEQ ID NO: 7.

It is also possible to conceive of variants in which the peptide contains 11, 12, 13, 14, 15, 16, 17, 18, 19, 20 or more peptides of SEQ ID NO: 1, SEQ ID NO: 2, SEQ ID NO: 3, SEQ ID NO: 4, SEQ ID NO: 5, SEQ ID NO: 6 and/or SEQ ID NO: 7.

Particular preference is given to dimers of the sequences having SEQ ID NO: 1, SEQ ID NO: 2, SEQ ID NO: 3, SEQ ID NO: 4, SEQ ID NO: 5, SEQ ID NO: 6 and/or SEQ ID NO: 7, both of the monomers being peptides having the same SEQ ID or having different SEQ IDs.

The peptide according to the invention is further preferably characterized in that the peptide substantially consists of D-amino acids.

In the context of the present invention, the term “substantially consists of D-enantiomeric amino acids” means that the monomers to be used according to the invention are formed to at least 50%, 55%, 60%, 65%, 70%, preferably 75%, 80%, particularly preferably 85%, 90%, 95%, in particular 96%, 97%, 98%, 99%, 100%, of D-enantiomeric amino acids.

The peptide according to the invention is further preferably characterized in that it consists of an amino acid sequence selected from the group consisting of SEQ ID NO: 1, SEQ ID NO: 2, SEQ ID NO: 3, SEQ ID NO: 4, SEQ ID NO: 5, SEQ ID NO: 6 or SEQ ID NO: 7.

The peptide according to the invention is further preferably characterized in that the peptide is linked to another substance.

In the context of the invention, the linking is a chemical bond as defined in Römpp Chemie Lexikon, 9th edition, volume 1, page 650 et. seq., Georg Thieme Verlag, Stuttgart; preferably a primary valency bond, in particular a covalent bond.

The substances are a variant of medicines or active ingredients defined according to the German Medicines Act § 2 or § 4 (19), September 2012 version. In an alternative, active ingredients are therapeutically active substances that are used as medicinally active substances. Use is preferably made of anti-inflammatories.

In a further variant, the substances are compounds that enhance the effects of the peptides.

In a further alternative, they are compounds that improve the solubility of the peptides and/or the passing of the blood-brain barrier.

In an alternative, according to the invention, the peptides have any desired combination of at least two or more features of the above-described variants, embodiments and/or alternatives.

The peptide according to the invention is further preferably characterized in that a plurality of peptides of SEQ ID NO: 1, SEQ ID NO: 2, SEQ ID NO: 3, SEQ ID NO: 4, SEQ ID NO: 5, SEQ ID NO: 6 or SEQ ID NO: 7 are covalently or non-covalently linked to one another.

In the context of the invention, a covalent bond or linkage of the peptide units is present if the peptides are linked linearly to one another either head-to-head, tail-to-tail or head-to-tail, with or without linker groups inserted therebetween.

The peptide according to the invention is further preferably characterized in that a plurality of peptides of SEQ ID NO: 1, SEQ ID NO: 2, SEQ ID NO: 3, SEQ ID NO: 4, SEQ ID NO: 5, SEQ ID NO: 6 or SEQ ID NO: 7 are linked to one another without a linker, i.e. directly linked to one another, or are linked to one another using a linker group.

In the context of the invention, a non-covalent linkage is present if the peptides are linked to one another for example via biotin and streptavidin, in particular streptavidin tetramer.

The peptide according to the invention is further preferably characterized in that a plurality of peptides of SEQ ID NO: 1, SEQ ID NO: 2, SEQ ID NO: 3, SEQ ID NO: 4, SEQ ID NO: 5, SEQ ID NO: 6 or SEQ ID NO: 7 are linked to one another in a linear or branched manner.

In a variant of the present invention, the peptides may be linearly linked to one another, in particular as described above. In another variant, the peptides are linked to one another in a branched manner to give the peptide according to the invention.

According to the invention, a branched peptide may be a dendrimer in which the monomers are covalently or non-covalently linked to one another.

Alternatively, the peptides can also be linked to a platform molecule (for example PEG or sugar) and thus form a branched peptide.

Alternatively, combinations of these options are also possible.

In one configuration of the invention, the binding affinity of the peptides is defined by the dissociation constant (K_{D} value).

In one advantageous configuration of the invention, the dissociation constant (K_{D} value) of a peptide according to the invention is advantageously lowered. This is associated with improved properties of the peptides according to the invention, such as higher binding affinity and higher degradation efficiency and/or prevention of the formation of toxic α-synuclein oligomers or aggregates.

In a variant of the invention, peptides are used which bind to an α-synuclein monomer with a dissociation constant (K_{D} value) of at most 500 μM, preferably 250, 100, 50 μM, particularly preferably 25, 10, 1 μM, particularly preferably with a dissociation constant (K_{D} value) of at most 500 nM, 250, 100, 50, particularly preferably 25, 10, 1 nM, 500 pM, 100, 50, 25, 20, 15, 10, 9, 8, 7, 6, 5, 4, 3, 2, 1 pM down to the sub-pM range, it being possible to adopt any intermediate value.

The peptides according to the invention are further preferably characterized in that they bind to the α-synuclein peptide, preferably the unfolded α-synuclein peptide, with a K_{D} of less than 50 μM.

The peptides can for example be produced by chemical synthesis or peptide synthesis.

The peptide as claimed in any one of the preceding claims, for preventing the formation of α-synuclein peptide oligomers and/or α-synuclein peptide aggregates.

In a further variant, the peptide is a peptide for inhibiting or preventing the formation of α-synuclein peptide oligomers and/or α-synuclein peptide aggregates.

In a further variant, the peptide is a peptide for the detoxification of α-synuclein peptide oligomers and/or α-synuclein peptide aggregates.

The peptides according to the invention detoxify the α-synuclein peptide oligomers and/or α-synuclein peptide aggregates or polymers formed therefrom, such as fibrils, preferably by not binding thereto but rather bonding to α-synuclein monomer and, by shifting the equilibrium, lead to the reduction of the α-synuclein oligomers, thereby preventing them from being converted to toxic compounds. Accordingly, another subject of the present invention is a method for the detoxification of α-synuclein oligomers and aggregates or fibrils formed therefrom.

The inhibition or prevention of the formation of α-synuclein peptide oligomers and/or α-synuclein peptide aggregates, and the detoxification of the α-synuclein peptide oligomers and/or α-synuclein peptide aggregates can take place in vitro or in vivo.

The present invention also relates to a peptide as described above for use in the treatment of synucleinopathies.

The present invention also relates to a peptide as described above, in particular for use in the treatment of Parkinson's disease (PD), dementia with Lewy bodies (DLB) and multiple system atrophy (MSA), which are particularly associated with the misfolding and aggregation of the protein α-synuclein in certain cells.

The present invention also relates in particular to a peptide as described above for use in the treatment of Parkinson's disease (PD).

### BRIEF DESCRIPTION OF THE DRAWINGS

In the drawings:

FIG. 1 shows graphs representing the results of the Thioflavin-T assay with recombinant full-length L-enantiomeric α-syn and D-enantiomeric 16-mer peptides described below.

FIG. 2 is a graph representing the results of a MST measurement of SVD-1 and α-syn described below.

FIG. 3 is a graph representing the results of a multicycle SPR experiment of WT alpha-synuclein and SVD-1 described below.

FIG. 4 is a graph representing the results of an α-syn oligomer elimination experiment with SVD-1 described below.

FIG. 5 shows graphs representing the results of a substoichiometric inhibition of α-syn aggregation in the ThT assay described below.

FIG. 6 shows graphs representing the results of the gradient centrifugation of α-syn aggregation samples in the presence and absence of SVD-1 described below.

FIG. 7 is a graph representing the results of the Thioflavin-T assay with recombinant full-length L-enantiomeric-syn and D-enantiomeric SVD-1a described below.

FIG. 8 is a graph representing the results of the single cycle SPR experiment of WT α-syn and SVD-1a, SVD-6a or SVD-10a described below.

FIG. 9 shows graphs representing the results of the de novo and seeded ThT assay with SVD-1 and SVD-1_scrambled described below.

FIG. 10 shows graphs representing the results of the MST measurement of the interaction of SVD-1 and SVD-1a with full-length α-syn in PBS, pH 7.4, at 25° C. described below.

FIG. 11 shows graphs representing the results of the kinetic single-cycle assay with α-syn and immobilized SVD-1 and SVD-1a described below.

FIG. 12 shows graphs representing the results of the NMR analysis of ¹⁵N-labeled α-syn in interaction with SVD-1a (not isotope-labeled) described below.

FIG. 13 is a graph representing the results of the size-exclusion chromatography of monomeric α-syn after incubation with increasing concentrations of SVD-1a described below.

FIG. 14 shows graphs representing the results of the Thioflavin-T de novo aggregation of monomeric α-syn with SVD-1a and SVD-1_scrambled+5r described below.

FIG. 15 shows graphs representing the results of the de-novo aggregation analysis of α-syn in the presence and absence of SVD-1a, using ThT, DGC, CD and AFM described below.

FIG. 16 shows graphs representing the results of the ThT assay with SVD-1a and SVD-1_scrambled+5r as seed described below.

FIG. 17 shows graphs representing the results of the decomposition of PFF oligomers into α-syn monomers by SVD-1a described below.

FIG. 18 shows graphs representing the results of the analysis of the aggregation assay in the presence and absence of SVD-1a by means of ThT assay and CD spectroscopy described below.

FIG. 19 represents the results of the inhibition of the aggregation of seeded α-syn in cells by SVD-1a described below.

FIG. 20 is a graph representing the results of the increase in cell viability by SVD-1 and SVD-1a in the MTT assay with PFF oligomer seeds described below.

### DETAILED DESCRIPTION OF EMBODIMENTS OF THE INVENTION

The invention will be described in more detail below using nonlimiting examples.

### EXAMPLES

The peptides SVD-1, SVD-6, SVD-10 and SVD-14, SVD-1a, SVD-6a, SVD-10a were investigated more closely.

### Materials and Methods

### Recombinant Expression and Cleaning of Monomeric WT and α-syn A140C

N-terminally acetylated WT α-syn (hereinafter referred to as α-syn) and acetylated α-syn-A140C were expressed in E. coli BL21(DE3) which carries codon-optimized α-syn in the pT7 vector and the pNatB vector with the N-terminal acetylation enzyme from Schizosaccharomyces pombe. Expression took place in LB- or ¹⁵N-saturated M9 minimal medium with 1 mM IPTG after reaching an OD₆₀₀ of 1.2 and subsequent incubation for 4 h at 37° C. The pellet from 1 l of expression was resuspended in 25 ml 20 mM Tris, pH 8.0, and boiled at 95-100° C. for 2×15 min. After centrifugation at 20 000×g for 30 min at 4° C., the supernatant was precipitated with a final concentration of 0.45 g/ml ammonium sulfate. The protein was pelletized at 20 000×g for 30 min and resuspended in 50 ml 20 mM Tris-HCl, pH 8.0. After sterile filtration, the sample was loaded onto a HiPrep QFF 16/10 (Cytiva, USA, CV=20 ml) anion exchange column. The gradient elution was performed with a target concentration of 800 mM NaCl over 20 CV. Recombinant α-syn eluted at a conductivity of 28-32 mS/cm. The fractions containing recombinant α-syn were pooled and precipitated with ammonium sulfate as described above. The pellets were resuspended in 5 ml 50 mM Tris-HCl, pH 7.4, 50 mM NaCl and loaded onto a HiLoad Superdex 60/75 pg gel filtration column (Cytiva, USA, CV=120 ml). Expression gave 20-30 mg/l, determined through A275 with an extinction coefficient of 5600 M⁻¹ cm⁻¹. The protein aliquots were frozen with liquid nitrogen and stored at −80° C.

### D-Enantiomeric Peptides

D-enantiomeric peptides with C-terminal amidation were acquired from CASLO (CASLO, DK) as a lyophilized chloride salt powder having a purity of >95%. The peptides, e.g. SVD-1 and SVD-1a, were tested under different buffer conditions including PBS pH 7.4, with UV-vis absorbance measurements after 1 h of incubation at 37° C. and 20 800×g centrifugation showing that both compounds remained completely in the supernatant up to a starting concentration of at least 1 mM.

### Thioflavin T Assay

The thioflavin T (ThT) assay is customarily used for visualizing α-syn fibrillation, since the stain ThT is able to bind to the amyloidogenic cross-β-pleated sheet portions of the fibril structures. Recombinant α-syn was thawed over ice and centrifuged for 30 minutes at 21 000×g and 4° C. The concentration of the supernatant was determined. Lyophilized D-peptides were thawed for 1 h at RT and dissolved in 500 μl PBS, pH 7.4. After 30 minutes of centrifugation at 21 000×g, the concentration of the supernatant was determined by means of UV-vis, using the corresponding extinction coefficient at A₂₈₀. All ThT assay experiments were carried out at 37° C. with 15 μM ThT and 0.05% sodium azide (w/v) in PBS, pH 7.4, unless stated otherwise. The ThT fluorescence was monitored with a baseline optical configuration at λₑₓ=448 nm and λₑₘ=482 nm in a fluorescence plate reader with orbital averaging to 3 mm (Clariostar or Polarstar Optima, BMG labtech, GE). Before measurement began, 120 μl of the sample solution was added to a non-binding 96-half area well plate with transparent flat base (Corning, USA). For all de novo aggregation assays, a borosilicate glass bead (d=3.0 mm, Hilgenberg, GE) was added to each well. For inoculated aggregation assays, no bead was used and the samples were incubated under resting conditions. The wells surrounding the sample wells were filled with the same volume of buffer in order to improve heat distribution. Unless stated otherwise, the experiments were performed with five repeats (n=5).

The ThT assay was used for different purposes. Firstly, the de novo ThT aggregation assays served as a screening platform for the aggregation lag with the synthetic D-peptides. In this case, 50 μM of recombinant α-syn was incubated with a three-times molar excess of each D-peptide. The samples were shaken before every cycle in orbital shaking mode at 300 rpm for 30 s. Peptides which were insoluble in aqueous buffer were dissolved in 2.5 μl DMSO (0.5 mg peptide) and gradually mixed with PBS, PH 7.4, until a final concentration of 2.5% (v/v) DMSO was reached. For these samples, the reference aggregation of α-syn alone in PBS, pH 7.4 with 2.5% (v/v) DMSO was also carried out. The aggregation curves of each replicate were adjusted individually using a symmetrical Boltzmann sigmoidal fit (OriginPro 2020, OriginLab, USA) using the following formula

$$y = \frac{{A}_{1} - {A}_{2}}{1 + {e}^{( x - {x}_{0} ) / dx}} + {A}_{2}$$

$$( {A}_{1} = starting value , {A}_{2} = end value , {x}_{0} = inflection point [ s ] , dx = time constant [ 1 / s ] ) .$$

The inflection point of the adjustment determines the aggregation half-life t_{1/2}, while the lag time was approximated using the following formula: t_{1/2}−2·dt_{1/2}, where dt_{1/2} is defined as the gradient of the adjustment at x=t_{1/2} in 1/s. The half-life and the lag time were calculated as mean values of the individual adjustments. In order to determine the concentration dependency of the aggregation lag, different concentrations of the compound were applied to 50 μM of recombinant α-syn. The lag time and t_{1/2} were calculated as described above. For the graphical representation, the adjusted steady states were normalized to 1 for all conditions, and the mean error was calculated on the basis of the adjustments. Statistical tests regarding the significance of the time shifts were carried out using Welch's t-test for two random samples, with p<0.05 (OriginPro 2020, OriginLab, USA). For de novo ThT assays with substoichiometric substance concentrations, 10 μM α-syn were used. In contrast to the screening aggregation assay, the α-syn samples were continuously shaken at 300 rpm in orbital shaking mode in order to shorten the aggregation time, with measurements being taken every 5 minutes. Statistical evaluation took place as described above, by comparing the significance of t_{1/2} on the lag time shifts and on steady state reduction in the presence of the inhibitor and the same concentration of the corresponding control peptide. For inoculated ThT assays, 50 nM of monomer-equivalent PFF oligomers were incubated in the fluorescence plate reader at 37° C. under resting conditions, together with or in the absence of different concentrations of the compounds. After 20 hours, 20 μM of monomeric α-syn were added to the sample mixture in order to begin seeding. The measurements were carried out every 5 minutes (n=3).

### Production of PFF Oligomers

PFF oligomers were produced as follows. Firstly, insoluble PFF was produced by incubating 300 μM of recombinant α-syn in a LoBind reaction tube (Eppendorf GmbH, GE) with a borosilicate glass bead (d=3.0 mm; Hilgenberg, DE) in 20 mM NaPi, pH 7.0, 150 mM 0.05% NaCl (w/v) sodium azide for one week at 37° C. The insoluble PFF was harvested by ultracentrifugation at 100 000×g for 30 min at 4° C., and the pellet was washed several times with 20 mM NaPi, PH 7.0, 150 mM NaCl. The monomer equivalent concentration was determined by measuring the α-syn concentration in the supernatant after the first centrifugation and subtracting this from the start concentration for the fibrillation. The insoluble PFF was resuspended in buffer and frozen at −80° C. with liquid N₂. Oligomeric PFFs were obtained by aggressive sonication of 200 μl insoluble PFF with 300 μM of monomer-equivalent concentration for 3×15 s (1 sec start/finish) and 60% amplitude using a tip sonicator (MS 72 micro tip, Sonopolus, Brandelin, GE). Insoluble PFFs were separated off by centrifugation at 100 000×g for 1 h at 4° C. The supernatant with the PFF oligomers was separated off, aliquoted and frozen at −80° C. with liquid N₂.

### Fluorescence Labeling

The peptides were fluorescence labeled by means of cysteine-maleimide coupling. SVD-1_Cys and SVD 1a_Cys were fluorescence labeled at the C-terminus with a fivefold or tenfold molar excess of TCEP and CF633-PEG2-maleimide (Sigma-Aldrich, USA). The labeling reaction was carried out in 25 mM NaPi, PH 7.0, for 2 h at RT. The labeled peptides were purified at 25° C. using an Agilent 1260 Infinity II System and a C-18 RP-HPLC column (Zorbax 300 SB-C8/SB-C 18, Agilent, USA). The mobile phases consisted of A: water+0.1% TFA, and B: acetonitrile+0.1% TFA. The fluorescence-labeled peptides and proteins were eluted with a gradient of 5-40% (v/v) B in 30 min. The fluorescence-labeled products were fractionated using the absorption wavelength of the fluorophore (CF633: 633 nm) and the pooled samples were subsequently lyophilized and redissolved in the appropriate buffer. The protein concentrations were determined by means of UV-vis and the fluorescence-labeled samples were stored at −20° C.

### Microscale Thermophoresis

The experiments for microscale thermophoresis (MST) were carried out using a Monolith NT.115 device (NanoTemper Technologies GmbH, GE). Fluorescence-labeled peptides were diluted in PBS, PH 7.4 to a final concentration of 100 nM. Recombinant α-syn was diluted in PBS, pH 7.4, in a series of serial dilutions from 50 μM to 3 pM final concentration and was mixed evenly with the peptide solutions. The samples were loaded into standard-surface-area glass capillaries for SVD1 and premium-surface-area capillaries for SVD-1a (NanoTemper Technologies GmbH, GE), and the measurements were carried out at 25° C. with 20% LED power and 60% MST power. The standard parameters recommended by the manufacturer were used (lag time of the heating period of 30 s, and re-equilibration period of 5 s). The data was evaluated with the thermophoresis effect, using the NT analysis software (version 1.5.41) supplied by the manufacturer.

### Surface Plasmon Resonance Kinetic Experiments

The measurements were carried out using an 8K-Biacore device (Cytiva, USA). The interactions were measured using single-cycle kinetics experiments. In all assays, the peptide compounds were immobilized as ligands on the sensor surface and recombinant α-syn was injected into the flow as the analyte. The peptides, in particular SVD-1 and SVD-1a, were immobilized via primary amino groups on a CMD200M carboxyldextran matrix chip (Xantec, GE). Immobilization took place after a 7-minute EDC/NHS activation at 10 μl/min with 50 μg/ml of peptide in 10 mM NaAc, pH 5.0 for SVD-1, and pH 7.0 for SVD-1a, until a saturation signal was reached (SVD-1: 400 RU, SVD-1a: 500 RU). Surface quenching was carried out with 1 M ethanolamine at pH 8.3. Unless stated otherwise, the kinetic experiments were carried out at a flow rate of 30 μl/min in PBS, pH 7.4. The surface was regenerated between cycles using 30 s injections of 2 M Gua-HCl at 30 μl/min. Data evaluation was performed using the evaluation software Biacore insight v3.0 (Cytiva, USA).

### Production of the Spin Label SVD-1a_Cys_MTSL

Analogs of SVD-1a spin-labeled with nitroxyl radicals were produced by reacting (1-oxyl-2,2,5,5-tetramethyl-Δ3-pyrroline-3-methyl) methanethiosulfonate (MTSL) (Toronto Research Chemicals, USA) with SVD-1a_Cys, leading to covalent bonding of the nitroxyl radical spin label MTSL to the C-terminal D-cysteine residue. A fivefold molar excess of MTSL relative to the peptide, i.e. ˜4 mg MTSL (˜15 μmol) was dissolved in 90 μl N,N-dimethylformamide (DMF) and mixed with 810 μl 200 mM HEPES/NaOH buffer, pH 7.6. The solution was then added to ˜5 mg (˜3 μmol) of lyophilized SVD-1a_Cys. After incubation for 1 to 2 hours at RT, the reaction mixture was used in a semi-preparative RP-HPLC on a C8 column (Zorbax-300 SB, Agilent, GE) with a connected HPLC system (Agilent 1260, Agilent, GE). The spin-labeled peptides SVD-1a_Cys_MTSL were purified using an aqueous acetonitrile (ACN) gradient (8% ACN, 0.1% trifluoroacetic acid (TFA) up to 60% ACN, 0.1% TFA in Milli-Q water within 40 minutes) at a throughput rate of 4 ml min⁻¹ at 25° C. and detection at 214 nm. Purified spin-labeled samples were collected, aliquoted, snap-frozen in liquid N₂ and freeze-dried under vacuum (LT-105, Martin Christ, GE). This described method enables complete spin labeling of SVD-1a_Cys_MTSL with a purity of ≥98%.

### NMR Spectroscopy

The samples were produced at final concentrations of 25 μM full-length ¹⁵N-labeled acetyl-α-syn in the absence (reference) and in the presence of an equimolar amount of SVD-1a (not isotope-labeled, therefore invisible to NMR) in PBS buffer, pH 7.4, with addition of 5% D₂O as internal reference. 2D-¹H-¹⁵N-HSQC spectra were recorded back-to-back with a Bruker AVANCE NEO spectrometer (Bruker, USA) at 1200 MHz proton Larmor frequency. The temperature of the experiments was 10° C. The spectral dimensions were 16.02 ppm (¹H)×30 ppm (¹⁵N), with 2048 points in the ¹H dimension and 256 increments in the ¹⁵N dimension, giving a recording time of 53 ms for the ¹H dimension and 35 ms for the ¹⁵N dimension. 32 scans, with a recovery lag of 1 s between the scans, were captured for each increment, giving a total test duration of 4.8 h per spectrum.

NMR Paramagnetic Relaxation Enhancement (PRE) data were captured with 25 μM full-length ¹⁵N-labeled acetyl-α-syn in the presence of 25 μM paramagnetically-labeled (but not isotopically enriched) SVD-1a_Cys_MTSL (using an MTSL spin label covalently bonded to the C-terminus of SVD-1a), giving a ratio of α-syn: SVD-1a of 1:1. The intensities (Ipara) were extracted from 2D-¹H-¹⁵N-Best-TROSY-NMR spectra captured at 600 MHz and 10° C., with 128 scans each per increment, giving a total test duration of 16 hours. Reference data were obtained by adding a 20-fold molar excess of ascorbic acid to the same sample, as a result of which the paramagnetic effect of the spin label was quenched and a diamagnetic reference sample was obtained. The diamagnetic reference spectra and intensities (Idia) were recorded back-to-back under the same conditions as for the paramagnetic samples.

The NMR datasets were processed using the Bruker TopSpin software (Version 4.1.1) and visualized using CcpNmr Analysis (v2.4.2) (50). To assess the changes in the chemical shift of the α-syn resonances in the presence of SVD-1a in comparison to the reference spectrum (without SVD1a), the peak positions were extracted using the CcpNmr Analysis software. From the residue-specific changes in chemical shift in the ¹H and ¹⁵N dimensions, an absolute change in chemical shift was calculated according to the following formula:

$$Δδ = \sqrt{0.5 ( {δ}_{H}^{2} + ( 0.14 {δ}_{N}^{2} ) )}$$

To analyze the PRE data, the resonance intensities of the paramagnetic sample and the peak intensities of the diamagnetic reference sample were extracted using CcpNmr Analysis.

### Size Exclusion Chromatography (SEC)

The elimination of PFF oligomers was assessed by SEC and the subsequent detection of the PFF oligomer and monomer peaks. PFF oligomers were incubated with or without SVD-1a in PBS, pH 7.4, for 3 days at 37° C. under resting conditions in low-retention Eppendorf tubes (Eppendorf, GE). Prior to injection, the samples were centrifuged for 2 min at 20 800×g and 100 μl of sample were injected onto an SEC-HPLC column (Bio SEC-3 300 Å, Agilent, USA) using an Agilent 1260 Infinity II system (Agilent, USA) at a flow rate of 1 ml/min and PBS, pH 7.4 as mobile phase. The proteins were detected at A214.

### Dynamic Light Scattering (DLS)

The measurements were carried out using a SpectroSize 300 131 (XtalConcepts, GE) device and a sample volume of 1 ml in a sealed quartz cuvette (Hellma Group, GE). The samples were incubated under resting conditions at 37° C. Prior to the measurements, all the samples were centrifuged for 30 minutes at 4° C. and 21 000×g in order to remove any possible impurities from the solution. Data points were recorded every 30 seconds for time-dependent DLS measurements. The diffusion coefficients were obtained from analysis of the decay of the autocorrelation function of the scattered intensity, and were used to determine the apparent hydrodynamic radii using the Stokes-Einstein equation.

### Atomic Force Microscopy (AFM)

The samples were produced by diluting to an α-syn monomer concentration of 1 μM. 5 μl were incubated on a freshly-cleaved mica surface, and dried. The surfaces were then washed three times with 200 μl ddH₂O and dried using a gentle stream of N₂. The measurements were carried out in a Nanowizard 3 system (JPK BioAFM-Bruker Nano GmbH, GE) in intermittent contact mode, with 2×2 and 5×5 μm and line scan rates of 0.5-2 Hz, under ambient conditions with a silicon cantilever and a tip with a nominal spring constant of 26 N/m, an average tip radius of 9±2 nm and a resonance frequency of approximately 300 kHz (Olympus OMCL-AC160TS-R3). The images were processed using the JPK data processing software (version spm-5.0.84). For the illustrated height profiles, a polynomial adjustment was subtracted from each scan line, first independently and then using a limited data range.

### Circular Dichroism (CD) Spectroscopy

Far-UV circular dichroism (CD) data were captured using a Jasco J-1100 spectropolarimeter (Jasco, GE). 350 μl of the sample replicates from the inoculated assays were pooled and filled into a high-precision quartz cuvette having a layer thickness of 1 mm (Hellma-Gruppe, GE). A scanning rate of 20 nm/min was adopted, with five accumulations per sample at far-UV wavelengths of 260 to 190 nm. The baseline was corrected by subtracting just the measurements for the buffer.

### Cell Assay for α-Synuclein Aggregation

A construct encoding full-length A53T-mutated human α-syn and fused with YFP at the C-terminus was synthesized and introduced into the pMK-RQ expression vector (GeneArt; Thermo Fisher Scientific, USA). The α-synA53T-YFP construct was sub-cloned using the restriction sites NheI (5′) and NotI (3′) in the vector pIRESpuro3 (Clontech; Takara Bio, JPN). HEK293T cells (American Type Culture Collection) were cultured in high-glucose Dulbecco's Modified Eagle's Medium (DMEM; Sigma-Aldrich, USA) which had 10% fetal calf serum (Sigma-Aldrich, USA), 50 units/ml penicillin, and also 50 μg/ml streptomycin (Sigma-Aldrich, USA) added thereto. The cells were cultured at 37° C. in a humidified atmosphere with 5% CO₂.

The cells plated in DMEM were transfected with Lipofectamine 2000 (Invitrogen; Thermo Fisher Scientific, USA). Stable cells were selected in DMEM with 1 μg/ml puromycin (EMD Millipore, USA). Monoclonal cell lines were produced by means of fluorescence-activated cell sorting of a polyclonal cell population in 96-well plates using a MoFlo XDP cell sorter (Beckman Coulter, USA). The clonal cell line B5 was finally chosen from 24 clonal cell lines and is named αSynA53T-YFP cells.

The peptides were incubated for 2 hours with 1.5% Lipofectamine 2000 in OptiMEM at room temperature. The α-synA53T-YFP cells were plated out into a 384-well plate with poly-D-lysine coating (Greiner, AT) at a density of 1000 cells per well with 0.1 μg/ml Hoechst 33342 (Thermo Fisher Scientific, USA), and the previously prepared transfection mixture was directly added to the cells in the wells.

In order to seed cellular aggregation of α-syn to α-synA53T-YFP cells, 30 nM of soluble α-syn PFF oligomers were incubated with 1.5% Lipofectamine in OptiMEM for 2 hours at room temperature, and 3 hours after the first transfection, were added to each well.

The plates were then incubated at 37° C. in a humidified atmosphere with 5% CO₂. On the third day, the cells were imaged in an IN Cell Analyzer 6500HS system (Cytiva, USA) using the blue and green fluorescence channels, and analyzed by the IN Carta Image Analysis software (Cytiva, USA) which had been used to establish an algorithm for identifying intracellular aggregates in living cells. Four wells were used for each condition and 16 images were recorded per well and analyzed by a fully-automated algorithm in order to prevent distortions. Statistical analysis was performed using one-way ANOVA and subsequent Dunnett's test for multiple comparisons (GraphPad Prism 9, GraphPad Software, USA). The error bars represent the standard deviation.

### Cell-Viability Assay; CellGlo-Test

The CellTiter-Glo Luminescent Cell Viability Assay (Promega GmbH, GE) was used to determine the number of viable cells in the culture, based on quantification of the ATP present, which is an indicator of metabolically active cells.

After the cells had been cultured for three days in 384-well plates, 35 μl of the medium was removed from the wells and 40 μl of the CellTiter-Glo reagent was added directly to each well. After mixing, luminescence was measured 10 minutes later using a Fluostar (BMG labtech, GE).

### Immunofluorescent Cell Staining

After three days of cell culture on 384-well plates, the cells were fixed for 15 minutes in 4% formaldehyde (Sigma-Aldrich, USA) in PBS (pH 7.4). After three rounds of washing with PBS, each lasting 5 minutes, the cells were permeabilized with 0.25% Triton X-100 (Sigma-Aldrich, USA) in PBS for 10 minutes.

After three further 5-minutes washes with PBS, the cells were blocked for 30 minutes with 1% bovine serum albumin (Sigma-Aldrich, USA) in PBS with 0.1% Tween 20 (Sigma-Aldrich, USA). The cells were stained with CF633 (Biotium, USA) fluorescent labeled antibodies at a concentration of 8 μg/ml in 1% bovine serum albumin in PBS with 0.1% Tween-20 for 1-3 h at room temperature and in the dark. The anti-α-syn antibody syn211 (Abcam, UK) was used to detect overall α-syn.

The anti-aggregation-α-syn antibody 5G4 (Sigma-Aldrich, USA) was used to detect oligomeric and fibrillary α-syn. The recombinant anti-α-syn (phospho S129) antibody EP1536Y (Abcam, UK) was used to detect α-syn phosphorylated at serine 129. After a subsequent three rounds of washing in PBS, each lasting 5 minutes, the cells in PBS were imaged using an IN-Cell Analyzer 6500HS system with 40× magnification (Cytiva, USA).

### Cell Viability Assay; MTT Assay

The potential for saving PC12 cells (Leibniz-Institut DSMZ, GE) from α-syn toxicity by adding SVD-1, SVD-1a or SVD-1_scrambled was measured in an MTT (3(4,5-dimethylthiazol-2-yl)-2,5-diphenyltetrazoliumbromide) cell viability assay.

PC12 cells (Leibniz-Institut DSMZ, GE) were cultured at 37° C. on a collagen A-coated (Biochrom GmbH, GE) tissue culture flask in RPMI 1640 medium, supplemented with 5% fetal calf serum and 10% horse serum in an atmosphere humidified to 95% with 5% CO₂. 10 000 cells per well in a volume of 100 μl were seeded into collagen A-coated 96-well plates (Thermo Fisher Scientific, USA and incubated for 24 hours at 37° C. and 300 rpm in a thermocycler.

Subsequently, final concentrations of 30 nM α-syn, either in the absence of prior incubation, or after prior incubation with 15 μM SVD-1, SVD-1_scrambled or 0.5 μM SVD-1a, were added to the cells. Furthermore, 15 μM of the peptides alone, cell media, buffer without peptide and 0.1% Triton X-100 (cytotoxic compound) were used as controls. After further incubation in a 95% humidified atmosphere with 5% CO₂ at 37° C. for 24 hours, the viability of the cells was measured using the Cell Proliferation Kit I (MTT) (Roche Applied Science, CH), following the manufacturer's protocol.

The MTT-formazan product was quantified by measuring the absorbance at 570 nm, corrected by subtracting the absorbance at 660 nm, in a FluoroStar Optima plate reader (BMG labtech, GE). All results were normalized to untreated cells grown only in medium. The significance test was carried out by one-way ANOVA with Bonferroni post-hoc analysis (OriginPro 2020, OriginLab, USA; n=4).

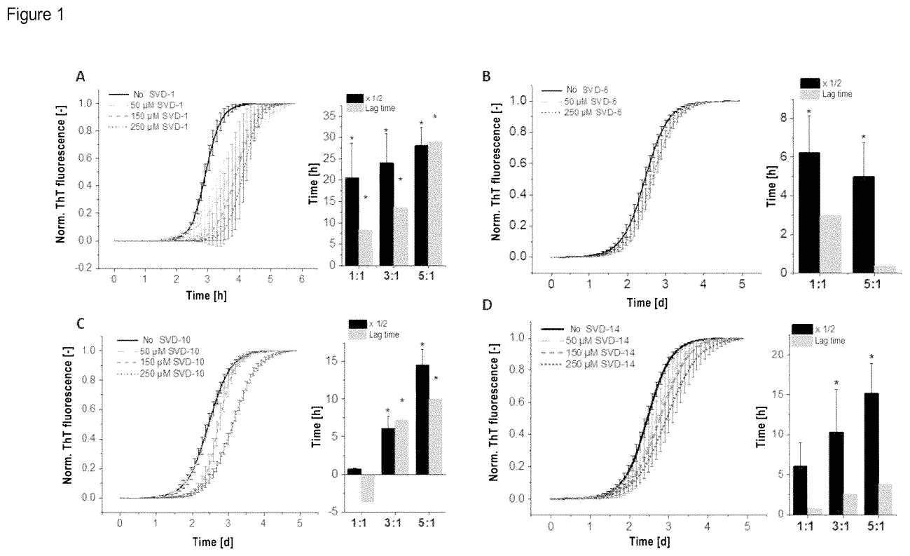

The results are shown in FIGS. 1 to 20.

FIG. 1: Thioflavin-T assay with recombinant full-length L-enantiomeric α-syn and D-enantiomeric 16-mer peptides.

50 μM α-synuclein (α-syn) were incubated with or without 50, 150 or 250 μM D-peptide in PBS, pH 7.4. The measurement was carried out in non-binding 96-well plates with 30 s of shaking at 300 rpm every 20 minutes at 37° C. One borosilicate glass bead (d=3 mm) was added to each well. The five replicates of each condition were individually fitted to the Boltzmann fit, normalized and the means were calculated together with the standard deviation (error bars). X ½ and lag-time shifts were determined by calculating the difference between samples with and without peptide. The significance of the time shifts was tested using a Welch's t-test for two random samples to be 0.05 (significant: *). (A) SVD-1, (8) SVD-6, (C) SVD-10, (D) SVD-14.

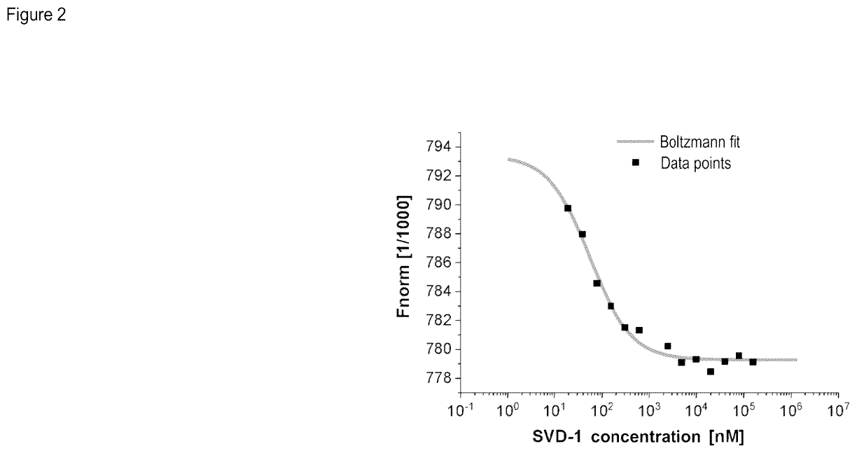

FIG. 2: MST measurement of SVD-1 and α-syn.

SVD-1 was incubated with concentrations of 159.5 μM to 19 nM and 30 nM 140C-C2-Alexa 647. 70% LED power; 60% MST power. The K_{D} was determined as 55 nM.

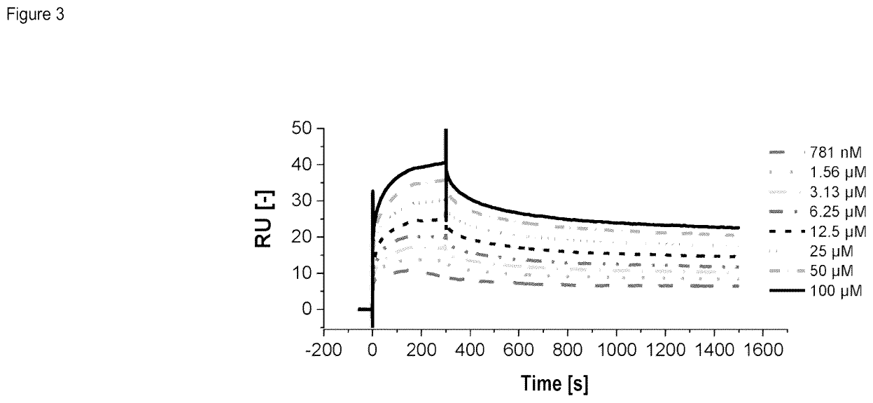

FIG. 3: Multicycle SPR experiment of WT alpha-synuclein and SVD-1.

400 RU of SVD-1 were immobilized on a 2D CMD sensor chip surface. WT α-syn was injected in a concentration range of 0.39-100 0.1 in PBS, PH 7.4. Regeneration: 2 M Gua-HCl and 0.1% SDS. The KD was determined by heterogeneous ligand adjustment, with K_{D}1 of 12.5 nM and K_{D}2 of 2.0 μM.

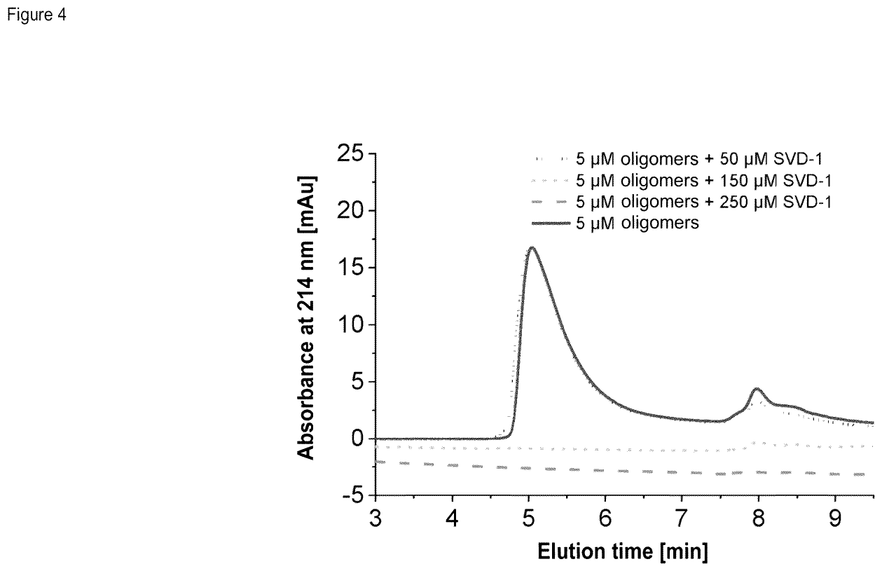

FIG. 4: α-syn oligomer elimination with SVD-1.

5 μM of monomer-equivalent oligomers were incubated with or without SVD-1 for 3 days at 900 rpm and 37° C. The samples were centrifuged at 21 000×g and the supernatant was loaded onto an HPLC-SEC column. It was not possible to detect any α-syn oligomer in the supernatant for SVD-1 concentrations of 150 and 250 μM.

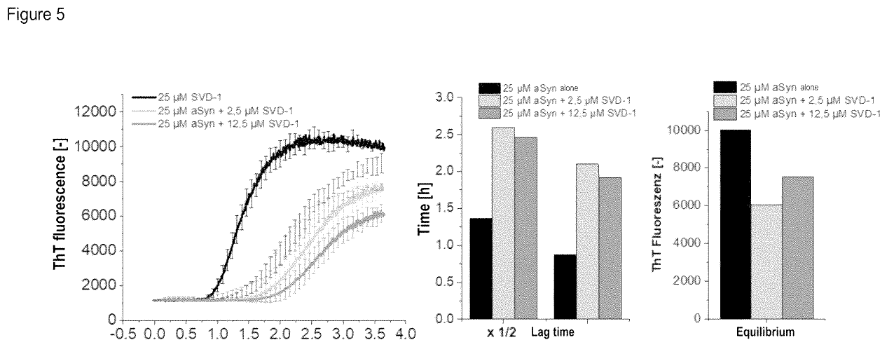

FIG. 5: Substoichiometric inhibition of α-syn aggregation in the ThT assay.

25 μM of monomeric α-syn was incubated with SVD-1 substoichiometric concentrations of 2.5 and 12.5 μM SVD-1. The measurement was carried out in non-binding coated 96 wells with continuous shaking at 37° C. One borosilicate bead (d=3 mm) was added per well. Each replicate was adjusted using a Boltzmann sigmoidal fit, and the mean values and lag time were calculated for each condition.

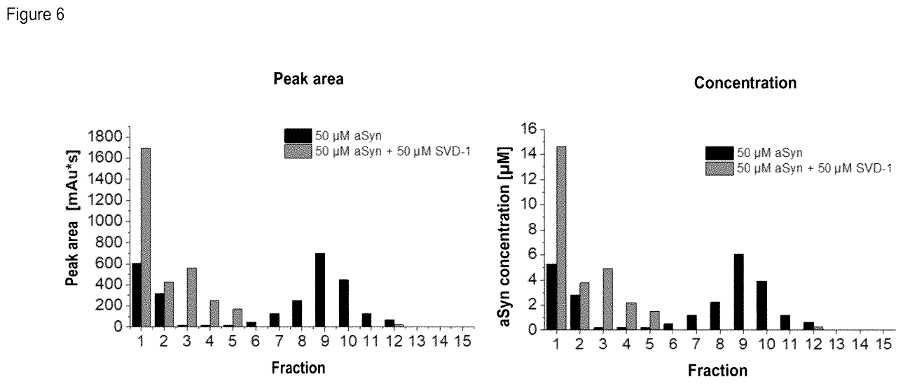

FIG. 6: Gradient centrifugation of α-syn aggregation samples in the presence and absence of SVD-1.

Monomeric α-syn was incubated with or without an equimolar concentration in SVD-1 in high-binding surface-coated 96-well plates (CORNING). No shaking, no glass beads, at 37° C. After 9 days, the triplet samples were combined together and 100 μl was added to an Iodixanol gradient (5-50% (w/v)). 15 fractions, each of 140 μl were analyzed by means of RP-HPLC using a Zorbax 300 SB-C8 column and integrated to α-syn peaks. No fibril formation (F6-F12) was observed in the presence of SVD-1.

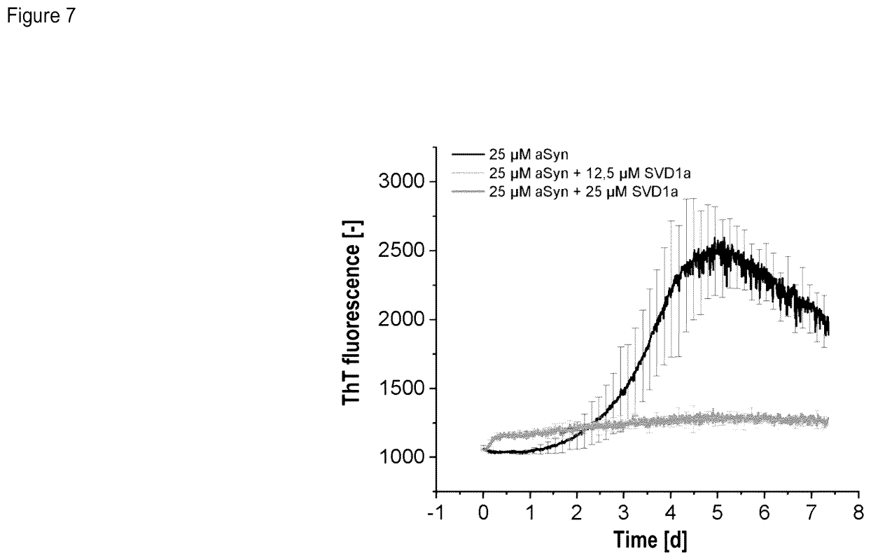

FIG. 7: Thioflavin-T assay with recombinant full-length L-enantiomeric α-syn and D-enantiomeric SVD-1a.

20 μM α-syn were incubated with or without substoichiometric concentrations of SVD-1a in PBS, pH 7.4. The measurement was carried out in non-binding coated 96 well-plates with continuous shaking at 400 rpm and 37° C. One borosilicate bead (d=3 mm) was added per well. For each condition, the mean value and the standard error of the five replicates was calculated.

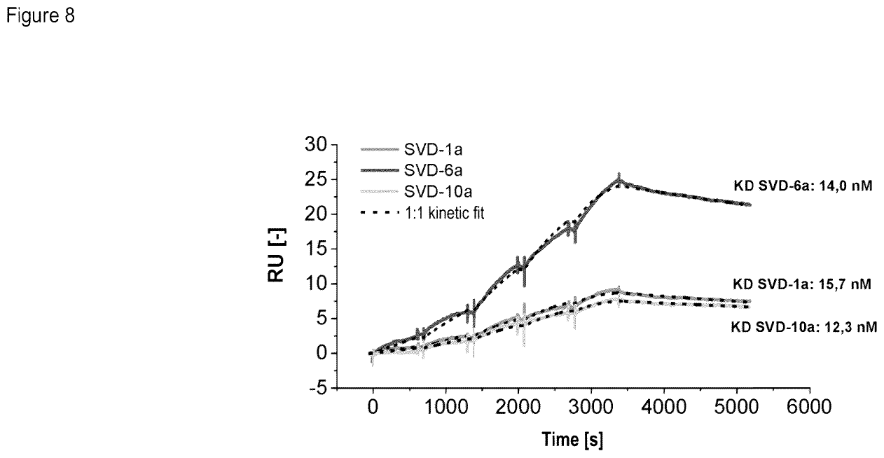

FIG. 8: Single cycle SPR experiment of WT α-syn and SVD-1a, SVD-6a or SVD-10a.

Each peptide was immobilized on a CM5 sensor chip surface. WT α-syn was injected in a concentration range of 30-500 nM in PBS, PH 7.4, 0.005% (v/v) Tween-20. Regeneration was carried out after every cycle with 2 M Gua-HCl for 2×30 sec. The K_{D}s were determined with a 1:1 binding model fit, as follows: SVD-1a: 15.7 nM; SVD-6a: 14.0 nM; SVD-10a: 12.3 nM.

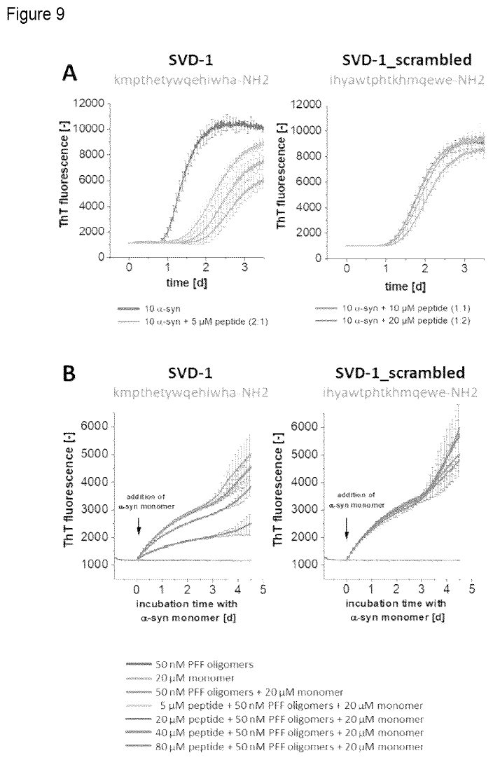

FIG. 9:

De novo and seeded ThT assay with SVD-1 and SVD-1_scrambled.

Both assays were carried out in a PBS buffer system at pH 7.4. The course of the ThT fluorescence was measured in a 96-well half-area plate (Corning, USA) using a Fluorostar plate reader (BMG labtech, GE) at λex=448 nm and λem=482 nm. The peptide sequences are shown in gray as the single-letter amino acid code.

(A) de novo aggregation assay with SVD-1 and SVD 1_scrambled. 10 μM α-syn monomer were incubated with 5, 10 and 20 μM peptide at 37° C., with one borosilicate glass bead (d=3.0 mm, Hilgenberg, GE) being added per well and with continuous shaking at 300 rpm between measurements. Mean data±SD (n=5) is reported.

(B) for the seeded aggregation, 50 nM of monomer-equivalent PFF oligomers were pre-incubated as seeds for 20 hours with or without peptide at 37° C. under resting conditions. Only then were 20 μM α-syn monomer added in order to induce seeding. Mean data±SD (n=3) is reported.

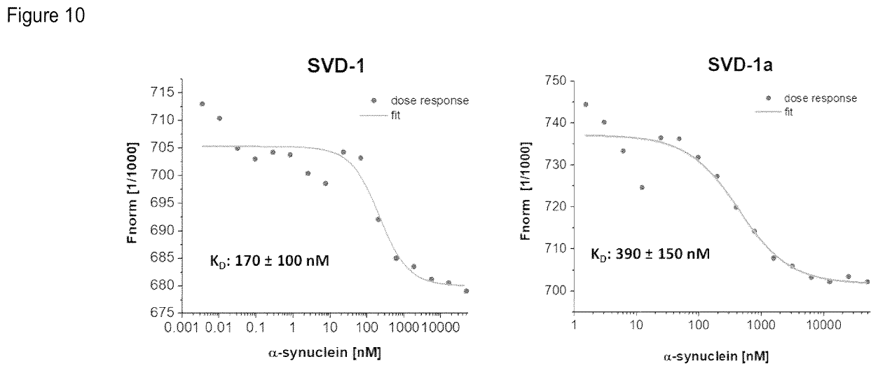

FIG. 10:

MST measurements of α-syn with SVD-1 and SVD-1a. MST measurement of the interaction of SVD-1 and SVD-1a with full-length α-syn in PBS, PH 7.4, at 25° C.

α-syn was diluted in a series of serial dilutions from 50 μM to 1 pM and was mixed with a final concentration of 100 nM fluorophor-labeled peptide (Alexa 647 C2). The experiment was carried out as an individual measurement. The K_{D} was determined to be 170 nM±100 nM for SVD-1 and 390 nM±150 nM for SVD-1a.

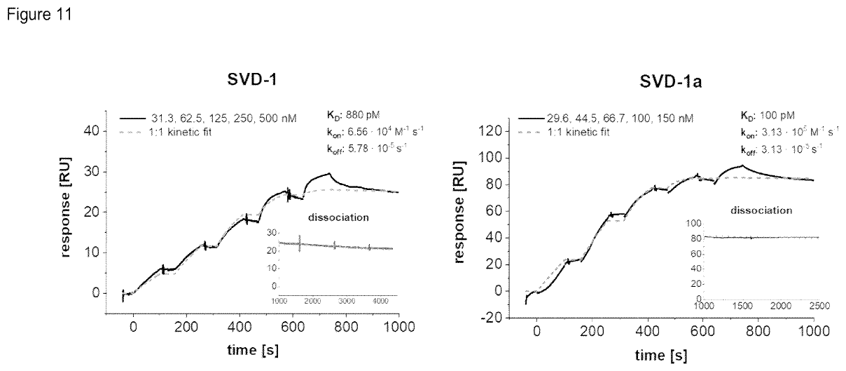

FIG. 11:

Kinetic single-cycle assay with α-syn and immobilized SVD-1 and SVD-1a.

SVD-1 and SVD-1a were immobilized on a carboxyldextran matrix by amino coupling to saturation (CMD200M, Xantec, GE). α-syn was injected for 100 s at 30 μl/min in PBS 7.4 in a serial dilution from 30 to 500 nM, followed by a dissociation time of 60 min or 30 min. The experiment was carried out as an individual measurement. The interaction kinetics were adjusted using a kinetic 1:1 interaction model: SVD-1: KD: 880 pM, Kₒₙ: 6.56·104 M⁻¹ s⁻¹, K_{off}: 5.78 s⁻¹·10⁻⁵; SVD-1a: KD: 100 pM, Kₒₙ: 3.13·105 M⁻¹ s⁻¹, K_{off}: 3.13·10⁻⁵ s⁻¹.

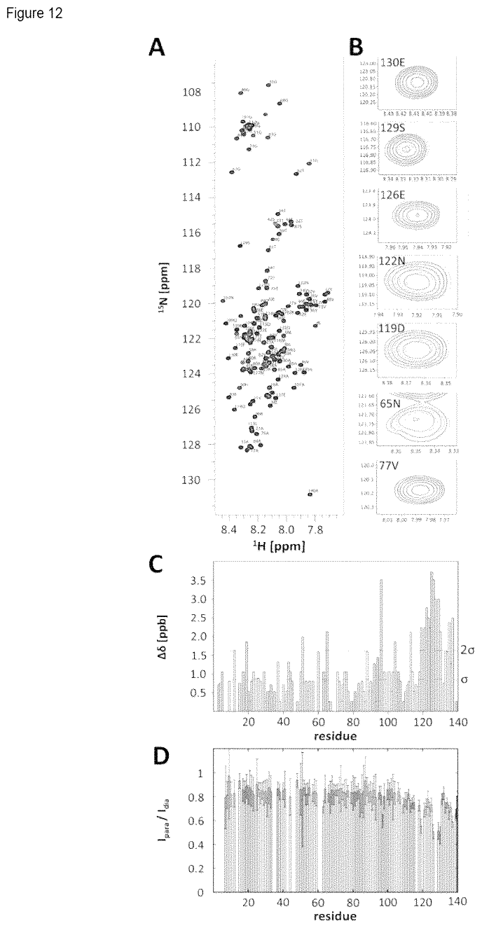

FIG. 12:

NMR analysis of ¹⁵N-labeled α-syn in interaction with SVD-1a (not isotope-labeled).

(A) overlay of two-dimensional ¹H-¹⁵N-HSQC spectra of 25 μM ¹⁵N-α-syn in the presence (red) and absence (black) of an equimolar amount of SVD-1a.

(B) enlargement of a number of resonances in the spectra shown in (A) which exhibit small chemical changes for residues in the presence (red) and absence (black) of SVD-1a (130E, 129S, 126E, 122N, 119D, 65N); for comparison, 77V resonance is shown which does not exhibit any change in chemical shift.

(C) residue-specific absolute NMR chemical shift changes in the spectra of ¹⁵N α-syn in the presence of SVD-1a, compared to in the absence of SVD-1a. The standard deviation σ, and double the standard deviation, 2σ, of the distribution of the observed changes in chemical shift are illustrated by dashed lines.

(D) NMR PRE intensity ratios of ¹⁵N α-syn in the presence of the paramagnetically-labeled SVD-1a. This shows the residue-specific intensity ratios, Ipara/Idia, of the cross-peak intensities in the two-dimensional ¹H-¹⁵N NMR spectra of the paramagnetic sample compared to the diamagnetic sample. The lower the Ipara/Idia intensity ratios, the closer the paramagnetically-labeled SVD-1a is to the α-syn residue in question. An intensity of one would indicate a lack of interactions. The data suggest a somewhat more pronounced (transient) bonding interaction of SVD-1a with residues in the C-terminal region of α-syn compared to the average effect on residues in the remaining N-terminal region.

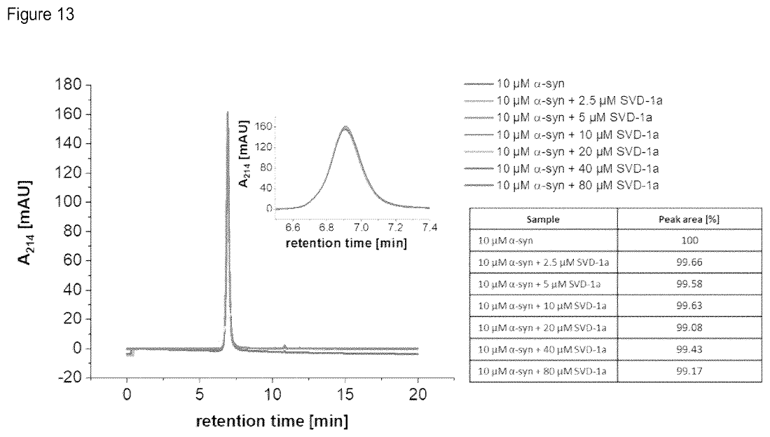

FIG. 13

Size-exclusion chromatography of monomeric α-syn after incubation with increasing concentrations of SVD-1a.

10 μM α-syn monomer were incubated with 0, 5, 10, 20, 40 and 80 μM SVD-1a in PBS, pH 7.4, for 2 h at 37° C. and 300 rpm. The SEC was carried out with a Bio SEC-3 column (150 Å, Agilent, USA) at 1 ml min⁻¹ and PBS, pH 7.4, as mobile phase. The samples were centrifuged for 5 minutes at 20 800×g before the supernatant was injected. The protein was detected at A214. α-syn monomer eluted after 6.91 min retention time. The peak area of the α-syn monomer was integrated for the 10 μM α-syn sample and set to 100%. The concentration of monomeric α-syn was not reduced in the presence of increasing concentrations of SVD-1a. SVD-1a itself was only eluted during the purification and regeneration procedure of the SEC, which is highly probably attributable to its high net positive charge.

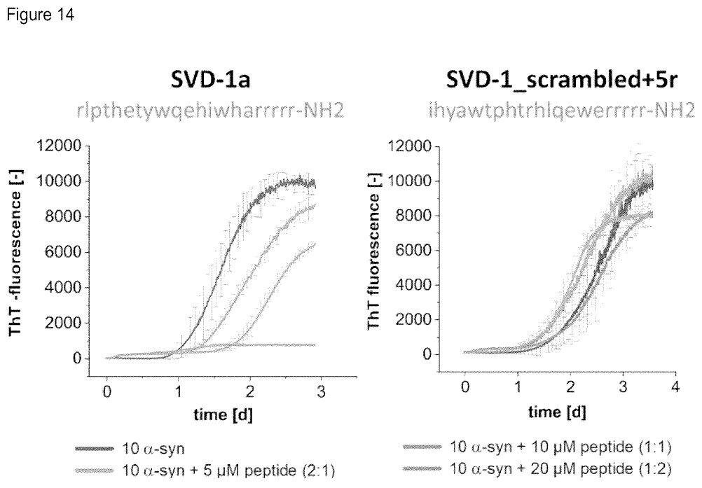

FIG. 14:

Thioflavin-T de novo aggregation of monomeric α-syn with SVD-1a and SVD-1_scrambled+5r.

The experiment was carried out in a PBS buffer system, pH 7.4. The course of the ThT fluorescence was measured in a 96-well half-area plate (Corning, USA) using a Fluorostar plate reader (BMG labtech, GE) at λₑₓ=448 nm and λₑₘ=482 nm. 10 μM α-syn monomer were incubated with 5, 10 and 20 μM peptide at 37° C., with one borosilicate glass bead (d=3.0 mm, Hilgenberg, GE) being added per well and with continuous shaking of the plates at 300 rpm between measurements. The data is reported as the mean±SD (n=5). The amino acid sequences of the peptides used are given in gray text.

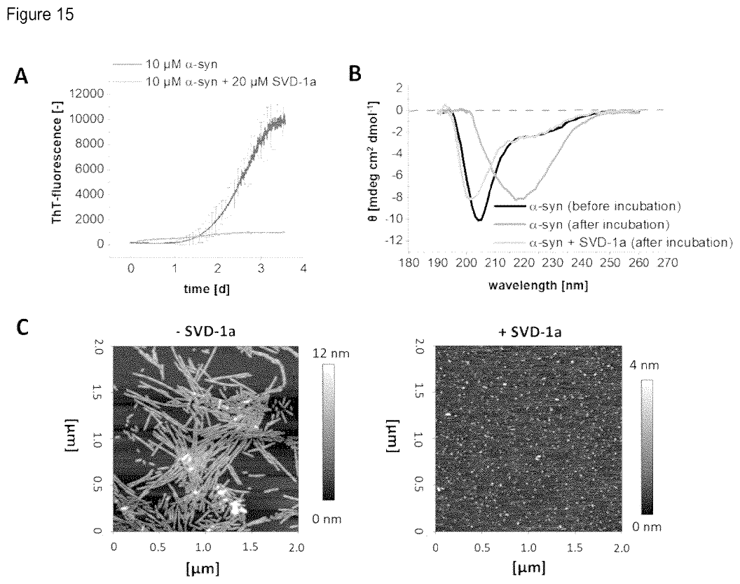

FIG. 15:

De-novo aggregation analysis of α-syn in the presence and absence of SVD-1a, using ThT, DGC, CD and AFM.

(A) De novo ThT assay of 10 μM α-syn with and without 20 μM SVD-1a. The course of the ThT fluorescence was measured in a 96-well half-area plate (Corning, USA) using a Fluorostar plate reader (BMG labtech, GE) at λₑₓ=448 nm and λₑₘ=482 nm, with continuous shaking of the plates at 300 rpm between measurements. The data is reported as the mean±SD (n=5).

(B) CD secondary structure analysis of de novo aggregate samples. The samples were incubated as described in (A) without adding any ThT (n=3) and were subsequently pooled for the CD analysis. The far-UV ellipticity of the samples was measured in a quartz cuvette (I=10 mm) in a J-1100 CD spectrometer (Jasco, GE). In addition to (A), a sample with 20 μM SVD-1a alone was incubated under identical conditions and later used as a reference for the samples with α-syn and SVD-1a. For these samples (α-syn+SVD-1a (after incubation)) the CD spectrum subtracted from the SVD-1a reference is shown.

(C) The samples from (A) were isolated directly after incubation and diluted in PBS, pH 7.4, to a final concentration of 1 μM α-syn monomer equivalent. 5 μl of the diluted sample were incubated and dried on a freshly cleaved mica surface, washed with ddH₂O thereafter, and dried using a gentle stream of N₂. The analysis was carried out using the NanoWizard 3 system (J-1100, JPK BioAFM, USA), with a plurality of surface sections being recorded. The sections shown in (C) are representative of the observed types identified in all surface sections.

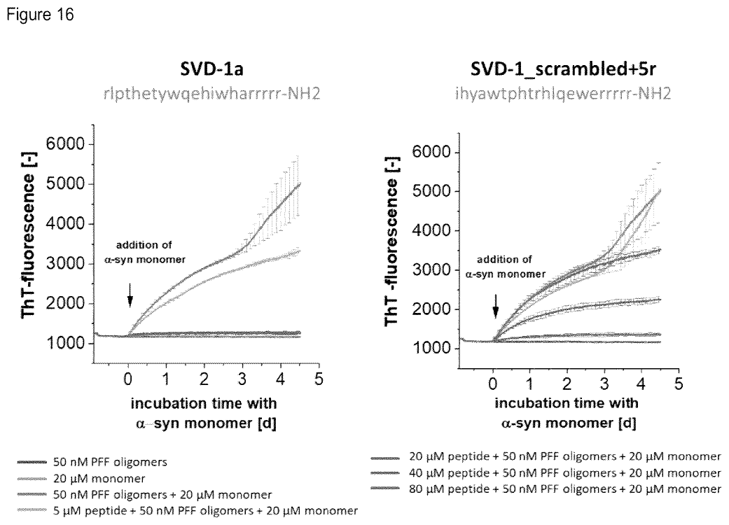

FIG. 16:

ThT assay with SVD-1a and SVD-1_scrambled+5r as seed.

The course of the ThT fluorescence was measured in a 96-well half-area plate (Corning, USA) using a Fluorostar plate reader (BMG labtech, GE) at λex=448 nm and λem=482 nm. The D-peptide sequences are given in gray as the single-letter amino acid code. 50 nM of monomer-equivalent PFF oligomers were pre-incubated as seeds for 20 hours with or without peptide at 37° C. under resting conditions. Only then were 20 μM α-syn monomer added to induce seeding, and to begin the incubation time with α-syn monomers. Mean data±SD (n=3) is reported.

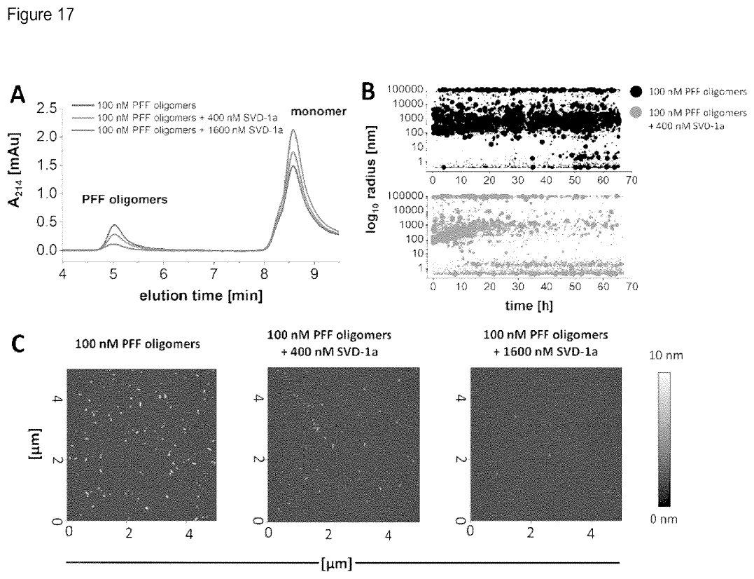

FIG. 17:

SVD-1a decomposes the PFF oligomers into α-syn monomers.

PFF oligomers were produced as described above. 100 nM monomer-equivalent PFF oligomers were incubated with or without 400 nM and 1600 nM SVD-1a for 3 days at 37° C. in PBS, PH 7.4.

(A) HPLC-SEC measurement samples were injected onto a Bio SEC-3 column (300 Å, Agilent, USA). The PFF oligomers eluted after approximately 5 min, while the α-syn monomer was detected after approximately 8.6 min.

(B) time-dependent DLS measurements with 100 nM PFF oligomers in the presence (red) and absence (black) of 400 nM SVD-1a. 1 ml of sample was continually measured in a sealed quartz cuvette at 37° C. under resting conditions every 30 s for 24 h in a SpectroSize 300 Instrument (XtalConcepts, GE). The data is shown as a radius diagram, with the signal amplitudes of each particle being represented by the diameter of the data point.

(C) For the AFM analysis, 5 μl of the samples described in (A) were taken off prior to centrifugation and incubated and dried on a freshly cleaved mica surface, subsequently washed with ddH2O and dried using a gentle stream of N2. The analysis was carried out using the NanoWizard 3 system (J-1100, JPK BioAFM, USA), with a plurality of surface sections being recorded. The sections shown in C are representative of the observed types and particle densities identified in all surface sections.

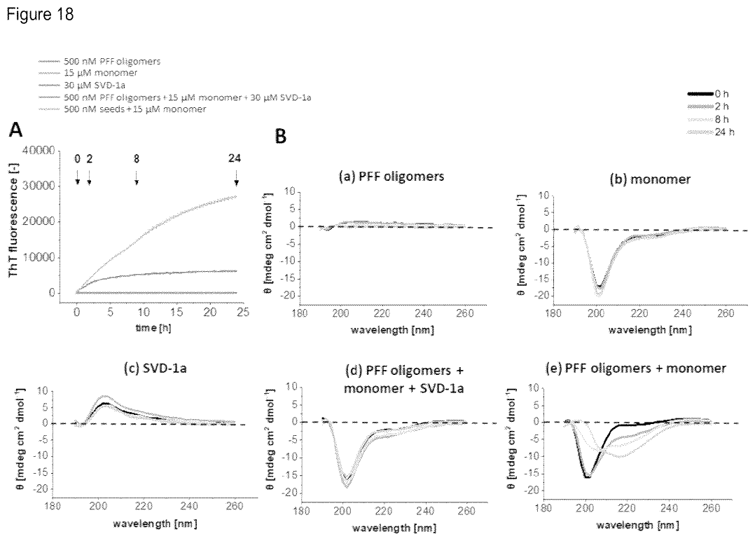

FIG. 18:

Analysis of aggregation assay in the presence and absence of SVD-1a by means of ThT assay and CD spectroscopy.

500 nM α-syn PFF were incubated at 37° C. for 24 h with or without 15 μM α-syn monomer and/or 30 μM SVD-1a in PBS, pH 7.4, under resting conditions.

(A) The course of the ThT fluorescence was measured in a 96-well non-binding half-area plate (Corning, USA) using a Fluorostar plate reader (BMG labtech, GE) at λₑₓ=448 nm and λₑₘ=482 nm. The data is reported as means±SD (n=3). The arrows indicate the points in time which were analyzed by CD spectroscopy.

(B) The samples shown in (A) were incubated under identical conditions without addition of ThT. The three replicates (120 μl each) for each condition were pooled and measured by means of CD spectroscopy at the points in time 0 h, 2 h, 8 h and 24 h. The far-UV ellipticity was measured in a quartz cuvette (I=10 mm) in a J-1100 CD spectrometer (Jasco, GE). The data shown for (d) were subtracted by (c) for the corresponding point in time.

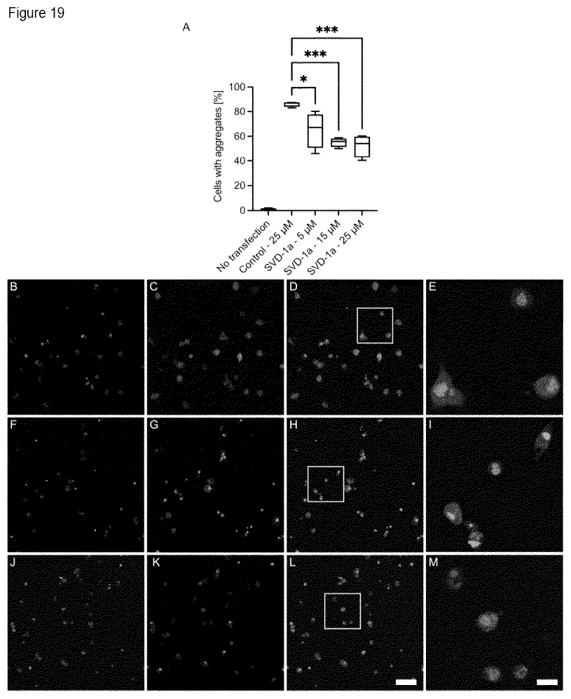

FIG. 19:

SVD-1a inhibits the aggregation of seeded α-syn in cells.

In order to validate the inhibitory effect of SVD-1a in cells, use was made of the α-synA53T-YFP cell system which stably expresses human α-syn with the familial A53T mutation, fused with YFP, in HEK293T cells, and the fluorescence-based detection of aggregates makes it possible to clearly show the strongly fluorescent spots within the cells against the background, intracellularly after seeding with PFF oligomers. In order to prevent the peptide from interfering with the assimilation of PFF oligomers, two-stage transfection was carried out, starting with a first transfection with peptide and a second transfection with PFF oligomers. SVD-1a, but not the peptide from the negative control (kgvgnleyqlwalegk-NH2), inhibited α-syn aggregation in αSynA53T-YFP cells in a dose-dependent manner (A). In order to quantify the number of cells with aggregates in our images and to prevent experimental distortions, use was made of a fully-automated algorithm for image analysis. The significance was calculated using one-way ANOVA and subsequent Dunnett's test for multiple comparisons. One asterisk represents a p value of less than 0.05 and three asterisks represent a p value of less than 0.001. The error bars represent the standard deviation. In contrast to non-seeded cells, which did not have any α-syn aggregates on the third day after seeding (B-E), seeding with soluble α-syn-PFF oligomers led to aggregation (F-I) in 85% of the cells treated with a peptide from the negative control (25 μM) that does not bind to α-syn. Treatment with increasing concentrations of SVD-1a, in this case 25 μM, led to a concentration-dependent reduction in the number of cells with aggregates (J-M). Panels B, E and H show nuclei stained with Hoechst 33342. Panels C, F and I show αSynA53T-YFP fluorescence. Panels D, G and J show composite images. The scale bar in L corresponds to 100 μm and applies to panels B-D, F-H and J-L. Panels E, I and M show enlargements of the insets in D, H and L. The scale bar in M applies to panels E, I and M and corresponds to 25 μM.

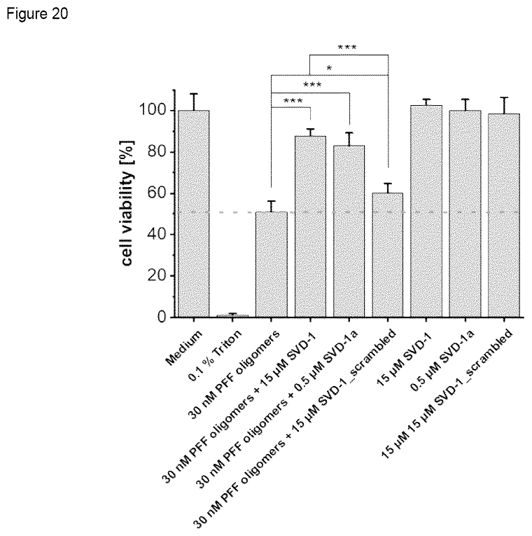

FIG. 20:

SVD-1 and SVD-1a increase cell viability in the MTT assay with PFF oligomer seeds.

SVD-1, SVD 1a SVD-1_scrambled were incubated overnight at 37° C. and 300 rpm with or without 30 nM PFF oligomers and were subsequently incubated with PC12 cells at final concentrations of 15 μM (SVD-1 and SVD-1_scrambled) or 0.5 μM (SVD-1a). While the viability of the cells with 30 nM PFF oligomers alone was reduced to 50%, 89 or 82% of the cells could be saved in the presence of SVD-1 or SVD-1a. A small increase in the cell viability-saving effect was also observed at 15 μM of the control peptide SVD-1_scrambled. The significance test was carried out by one-way ANOVA with Bonferroni post-hoc analysis: *p≤0.5, ***p≤0.01. The means are stated±SD (n=4).

What is claimed is:

1-15. (canceled)

16. A peptide, wherein the peptide comprises an amino acid sequence selected from SEQ ID NO: 1, SEQ ID NO: 2, SEQ ID NO: 3, SEQ ID NO: 4, SEQ ID NO: 5, SEQ ID NO: 6 or SEQ ID NO: 7, and homologs, fragments and portions thereof.

17. The peptide of claim 16, wherein peptide comprises an amino acid sequence selected from SEQ ID NO: 1, SEQ ID NO: 2, SEQ ID NO: 3, SEQ ID NO: 4, SEQ ID NO: 5, SEQ ID NO: 6 or SEQ ID NO: 7 or homologs having an identity of at least 80% thereto.

18. The peptide of claim 16, wherein at a free C-terminus, instead of a carboxyl group there is an acid amide group (CONH₂ group), a COH group, a COCl group, a COBr group, a CONH-alkyl radical or a CONH-alkylamine radical, or the peptide is present in cyclized form.

19. The peptide of claim 16, wherein the peptide contains 2 or more copies of sequences having SEQ ID NO: 1, SEQ ID NO: 2, SEQ ID NO: 3, SEQ ID NO: 4, SEQ ID NO: 5, SEQ ID NO: 6 or SEQ ID NO: 7.

20. The peptide of claim 16, wherein the peptide consists substantially of D-amino acids.

21. The peptide of claim 16, wherein the peptide consists of an amino acid sequence selected from SEQ ID NO: 1, SEQ ID NO: 2, SEQ ID NO: 3, SEQ ID NO: 4, SEQ ID NO: 5, SEQ ID NO: 6 or SEQ ID NO: 7.

22. The peptide of claim 16, wherein the peptide is linked to another substance.

23. The peptide of claim 16, wherein a plurality of peptides of SEQ ID NO: 1, SEQ ID NO: 2, SEQ ID NO: 3, SEQ ID NO: 4, SEQ ID NO: 5, SEQ ID NO: 6 or SEQ ID NO: 7 are covalently linked to one another.

24. The peptide of claim 16, wherein a plurality of peptides of SEQ ID NO: 1, SEQ ID NO: 2, SEQ ID NO: 3, SEQ ID NO: 4, SEQ ID NO: 5, SEQ ID NO: 6 or SEQ ID NO: 7 are non-covalently linked to one another.

25. The peptide of claim 16, wherein a plurality of peptides of SEQ ID NO: 1, SEQ ID NO: 2, SEQ ID NO: 3, SEQ ID NO: 4, SEQ ID NO: 5, SEQ ID NO: 6 or SEQ ID NO: 7 are linked to one another without a linker.

26. The peptide of claim 16, wherein a plurality of peptides of SEQ ID NO: 1, SEQ ID NO: 2, SEQ ID NO: 3, SEQ ID NO: 4, SEQ ID NO: 5, SEQ ID NO: 6 or SEQ ID NO: 7 are linked to one another by a linker group.

27. The peptide of claim 16, wherein a plurality of peptides of SEQ ID NO: 1, SEQ ID NO: 2, SEQ ID NO: 3, SEQ ID NO: 4, SEQ ID NO: 5, SEQ ID NO: 6 or SEQ ID NO: 7 are linked to one another in a linear manner.

28. The peptide of claim 16, wherein a plurality of peptides of SEQ ID NO: 1, SEQ ID NO: 2, SEQ ID NO: 3, SEQ ID NO: 4, SEQ ID NO: 5, SEQ ID NO: 6 or SEQ ID NO: 7 are linked to one another in a branched manner.

29. The peptide of claim 16, wherein the peptide is a dendrimer in which peptides of SEQ ID NO: 1, SEQ ID NO: 2, SEQ ID NO: 3, SEQ ID NO: 4, SEQ ID NO: 5, SEQ ID NO: 6 or SEQ ID NO: 7 are linked by a platform molecule.

30. The peptide of claim 16, wherein the peptide is capable of binding to an α-synuclein peptide with a KD of less than 50 μM.

31. The peptide of claim 16, wherein the peptide is capable of preventing a formation of α-synuclein peptide oligomers and/or α-synuclein peptide aggregates.

32. The peptide of claim 16, wherein the peptide is capable of detoxification of α-synuclein peptide oligomers and/or α-synuclein peptide aggregates.

33. A method of treating a synucleinopathy in a subject in need thereof, wherein the method comprises administering to the subject the peptide of claim 16 in an amount which is effective for treating the synucleinopathy.

34. A method of preventing a formation of α-synuclein peptide oligomers and/or α-synuclein peptide aggregates in a subject, wherein the method comprises administering to the subject the peptide of claim 16.

35. A method of detoxification of α-synuclein peptide oligomers and/or α-synuclein peptide aggregates in a subject in need thereof, wherein the method comprises administering to the subject the peptide of claim 16.


SEQUENCE LISTING (7 sequences)

SEQ ID NO: 1 - protein, 16 aa, Artificial Sequence
  D-Peptide
KMPTHETYWQEHIWHA

SEQ ID NO: 2 - protein, 16 aa, Artificial Sequence
  D-Peptide
YDWKQPMSASRFLAPW

SEQ ID NO: 3 - protein, 16 aa, Artificial Sequence
  D-Peptide
SSHWQQWNPPYWNTDS

SEQ ID NO: 4 - protein, 17 aa, Artificial Sequence
  D-Peptide
YDWKQPMSASRFLAPWR

SEQ ID NO: 5 - protein, 21 aa, Artificial Sequence
  D-Peptide
RLPTHETYWQEHIWHARRRRR

SEQ ID NO: 6 - protein, 21 aa, Artificial Sequence
  D-Peptide
YDWRQPLSASRFLAPWRRRRR

SEQ ID NO: 7 - protein, 21 aa, Artificial Sequence
  D-Peptide
SSHWQQWNPPYWNTDSRRRRR